(19) United States
(12) Patent Application Publication
(10) Pub. No.: US 2016/0262836 A1
(43) Pub. Date: Sep. 15, 2016

(54) SYSTEM COMPRISING A SURGICAL INSTRUMENT AND A FLUSHING ROD

(71) Applicant: DANNORITZER Medizintechnik GmbH & Co. KG, Tuttlingen (DE)

(72) Inventor: Axel DANNORITZER, Tuttlingen (DE)

(21) Appl. No.: 14/645,521

(22) Filed: Mar. 12, 2015

(51) Int. Cl. A61B 17/00

(52) U.S. Cl. CPC A61B 19/34; A61B 17/00234; A61B 2019/343; A61B 2017/00323

(57) ABSTRACT

The invention relates to a system comprising a surgical instrument with a hollow main rod which has an opening, and comprising a flushing rod provided for flushing the hollow main rod and for being inserted into the hollow main rod through the opening.

The invention further relates to a method for using such a system.

### BACKGROUND

The invention relates to a system comprising a surgical instrument with a hollow main rod which has an opening, and comprising a flushing rod provided for flushing of the hollow main rod and provided for being inserted into the hollow main rod through the opening.

Surgical instruments comprising a hollow main rod and an opening provided for introducing a stream of a flushing fluid into the hollow main rod for cleaning the hollow main rod are known in the art. Such surgical instruments are for example being embodied as retractors provided for holding back organs or tissues from a surgery area.

The invention has the objective of providing a system comprising a surgical instrument which has a hollow main rod, wherein the hollow main rod can be reliably and thoroughly cleaned. The objective is achieved by a system comprising the features of the main claim. Further advantageous embodiments and additional advantageous features are described in the dependent claims.

### SUMMARY

The invention proposes a system comprising a surgical instrument with a hollow main rod which has an opening, and comprising a flushing rod provided for flushing the hollow main rod and for being inserted at least partially into the hollow main rod through the opening.

A “flushing rod” is to be understood, in the context of this invention, as a rod which has a hollow portion and which is provided for introducing a stream of flushing fluid. “Provided” is to mean, in particular, specifically designed and/or equipped. By an object being provided for a certain function, it is in particular to be understood that the object fulfills and/or implements said certain function in at least one application state and/or operating state. “Provided for being inserted into the hollow main rod” is, in the context of this invention, intended to mean that the flushing is being provided to be at least partially inserted into an interior space of the hollow main rod. “Being at least partially inserted into an interior space of the hollow main rod” is intended to mean, in the context of this invention, that the flushing rod is, in at least one operating state of the system, to be inserted through the opening into the hollow main rod such that at least forty percent, preferably at least sixty percent and most preferably at least eighty percent of a total length of the flushing rod are situated within the hollow main rod. With the flushing rod, the flushing fluid can be guided reliably to defined positions in the hollow main rod to flush out contaminants from the insides of the hollow main rod. Furthermore, by using the flushing rod, a process of flushing the hollow main rod can be simplified. By the at least partial insertion of the flushing rod into the hollow main rod, the flushing fluid can be reliably guided within the hollow main rod to reach defined positions.

Furthermore, the invention claims a system wherein the flushing rod is provided to be inserted along at least twenty percent of a longitudinal extension of the hollow main rod. By this design, positions located far from the opening can be reliably flushed with the flushing fluid.

Furthermore, the invention claims a system, wherein the opening is substantially arranged at one end of the hollow main rod. Being “substantially arranged at one end of the hollow main rod” is intended to mean, in the context of this invention, to be arranged on a portion of the hollow main rod that extends from one end of the hollow main rod along a maximum of ten percent of an extension of the hollow main rod. By this design, an opening that is easy to use can be achieved.

Furthermore, the invention claims a system, wherein the hollow main rod comprises a main cavity oriented in at least one operating state at least substantially along a longitudinal extension of the hollow main rod, and wherein the opening is provided for insertion of the flushing rod in a direction which is at least substantially parallel to a main extension direction of the cavity. A “main cavity” is to be understood, in the context of this invention, as a cavity having a volume which is greater than any of the volumes of other cavities of the hollow main rod. “In at least one operating state at least oriented along a longitudinal extension” is intended to mean, in the context of this invention, that the hollow main rod of the surgical instrument may be provided to be deformable during an operation and/or to change its shape in a controlled manner during an operation and that, in at least one operating state, the hollow main rod of the surgical instrument has a shape, wherein at least seventy percent of a length of the main cavity are oriented along a main extension direction of the surgical instrument. A “direction at least substantially parallel to a main extension direction of the cavity” is to be understood, in the context of this invention, as a direction which has an angle with a straight line of five arc degree at most, advantageously of three arc degree at most and preferably of one arc degree at most, said straight line being parallel to the main extension direction of the cavity. In a most preferred embodiment, the direction is parallel to the straight line. A “main extension direction of the cavity” is to be understood, in the context of this invention, as a direction which is at least substantially parallel to the longitudinal extension of the hollow main rod and in which the main cavity has a greater extension than along other directions perpendicular to the main extension direction. By this design, a simple inserting movement of the flushing rod into the hollow main rod can be achieved.

Furthermore, the invention claims a system, wherein the surgical instrument comprises a knob which is provided for operating the surgical instrument and has an opening for inserting the flushing rod into the opening of the hollow main rod. The opening of the knob may be embodied as being closable. By this design, a simple insertion of the flushing rod into the hollow main rod can be achieved.

Furthermore, the invention claims a system, wherein cable pulls are arranged in the hollow main rod. By this design, an actuation device having a simple construction can be achieved. In addition, a clogging of the cable pulls by contaminants can be prevented via flushing.

Furthermore, the invention claims a system, wherein the surgical instrument comprises a tool end designed to change its shape upon being actuated by the cable pulls. The tool end is provided for fulfilling a function during a surgical procedure, for example a gripping or a spreading of organs, and has a shape which is adapted for fulfilling this function. By this design, a contamination of the tool from contaminants clogging the cable pulls can be prevented via flushing. Surgical instruments having a tool end designed to change its shape upon being actuated by the cable pulls, for example retractors, are well-known in the state of the art.

Furthermore, the invention claims a system, wherein the flushing rod comprises a hollow rod and a connection provided for connecting the hollow rod to a source of flushing fluid. A “connection” is to be understood, in the context of this invention, as a connecting piece for connecting the hollow rod to a flushing fluid source, for example, to a water conduit. By this design, a simple and secure connection with a flushing fluid source can be achieved.

Furthermore, the invention claims a system, wherein the connection has a funnel-like shape. By this design, a high pressure and a rapid flow of the flushing fluid can be achieved, thus enabling a high flushing efficiency and a thorough cleaning of the hollow main rod.

Furthermore, the invention claims a flushing rod of said system.

Moreover, the invention claims a surgical instrument of said system.

Furthermore, the invention claims a knob of said surgical instrument.

Furthermore, the invention claims a method for using said system, comprising a step in which the flushing rod is inserted into the hollow main rod.

Moreover, it is proposed that the flushing rod is inserted into the hollow main rod along at least twenty percent of a longitudinal extension of the hollow main rod. By this step, positions located far from the opening can be reliably cleaned.

Furthermore, the invention claims a method comprising a further step of introducing a flushing fluid into the hollow main rod via the flushing rod. By this step, a thorough cleaning of the hollow main rod can be achieved.

Furthermore, the invention claims a method comprising a further step in which the flushing is pulled out of the hollow main rod while maintaining a stream of flushing fluid. By this step, a thorough cleaning of the hollow main rod can be achieved. Different parts of the hollow main rod can be thoroughly cleaned in subsequent order. Moreover, the opening for inserting the flushing rod into the hollow main rod can be used as an outlet for the flushing fluid flushing pollutants out of the hollow main rod.

### DRAWINGS

Further advantages may be gathered from the following description of the drawings. In the drawing a preferred embodiment of the invention is shown. The drawings, the description and the claims contain a plurality of features in combination. The individual skilled in the art will purposefully also consider the features separately and will arrange them in further expedient combinations.

### BRIEF DESCRIPTION OF THE DRAWINGS

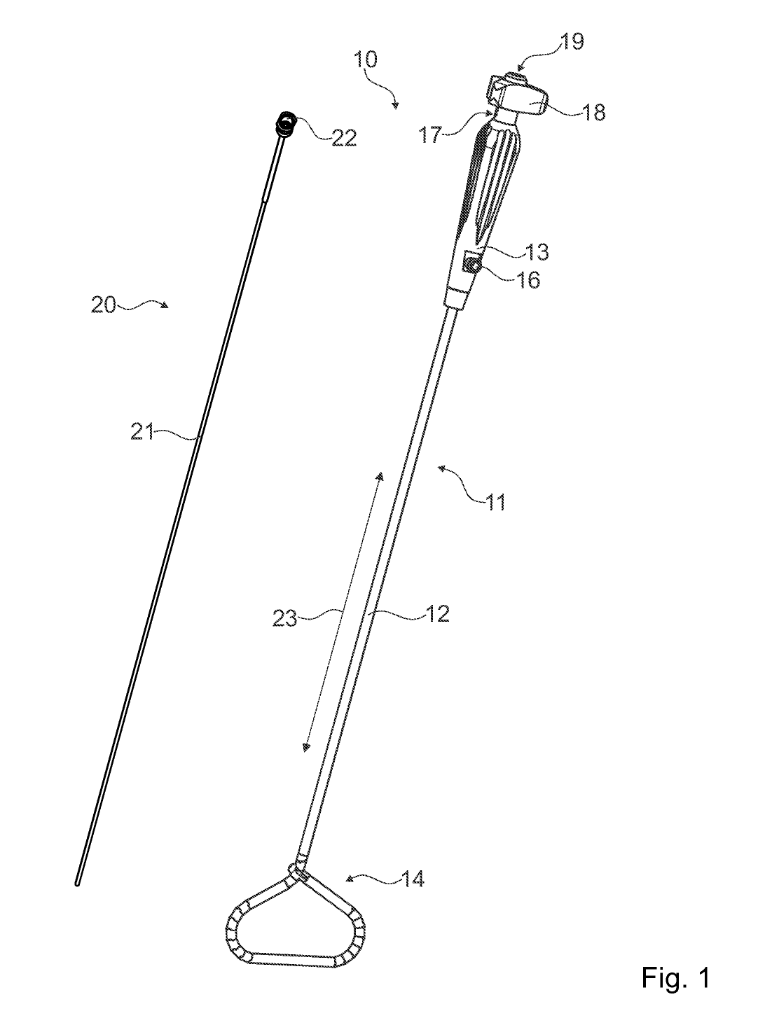

FIG. 1 shows a view of a system comprising a surgical instrument and a flushing rod with the flushing rod and the surgical instrument separated from one another,

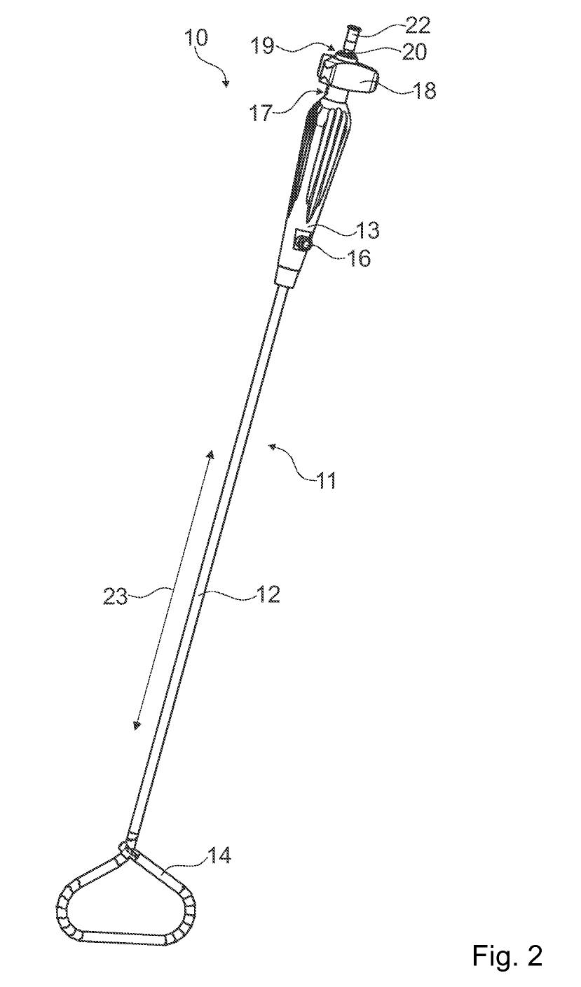

FIG. 2 shows a view of a system comprising a surgical instrument and a flushing rod with the flushing rod being inserted into the surgical instrument,

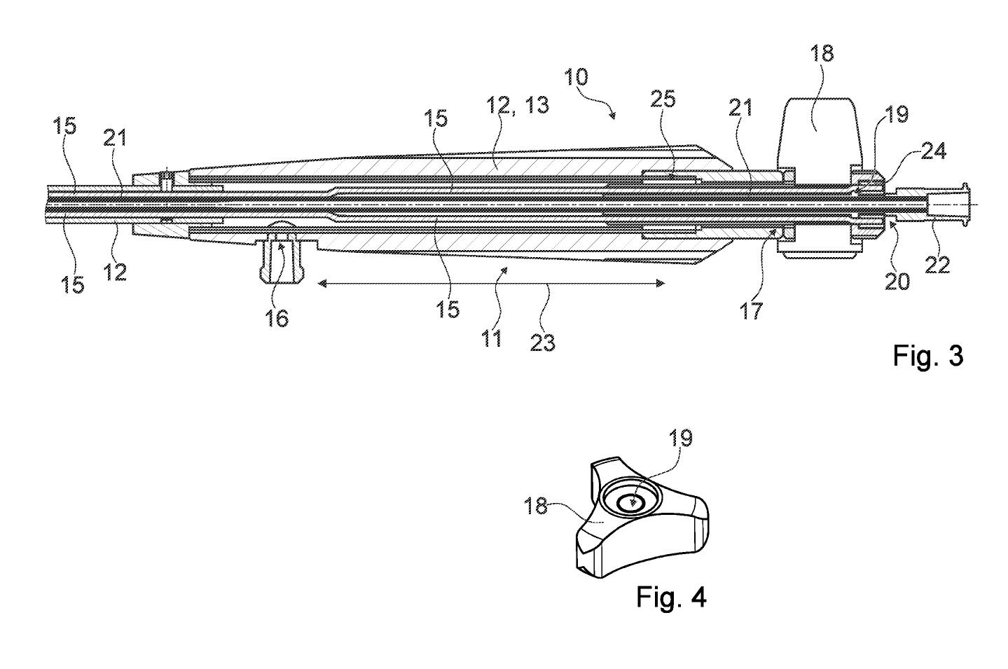

FIG. 3 shows an intersectional view of the system comprising a surgical instrument and a flushing rod with the flushing rod being inserted into the surgical instrument, and FIG. 4 shows a detailed view of a knob of the surgical instrument.

### DETAILED DESCRIPTION OF THE PREFERRED EMBODIMENT

FIGS. 1 to 4 show a system 10 comprising a surgical instrument 11 with a hollow main rod 12 which has an opening 17, and comprising a flushing rod 20 provided for flushing the hollow main rod 12 and for being inserted at least partially into the hollow main rod 12 through the opening 17. The flushing rod 20 can be inserted into the hollow main rod 12 with eighty percent of a total length of the flushing rod 20 being inserted into the hollow main rod 12. Such surgical instruments 11 are known in the state of the art.

The surgical instrument 11 comprises cable pulls 15 arranged in the hollow main rod 12. The surgical instrument 11 comprises a tool end 14 designed to change its shape upon being actuated by the cable pulls 15. The surgical instrument 11 is embodied as a retractor to retract organs and/or tissues during a surgical procedure, with the tool end 14 having a first shape, wherein the tool end 14 is in a predominantly straight shape to facilitate an insertion of the surgical instrument 11 into a body, and the tool end 14 having a second shape, wherein the tool end 14 forms a two-dimensional shape to retract an organ or tissue. The transition from the first shape to the second shape is actuated by twisting the cable pulls 15.

The flushing rod 20 is provided to be inserted along at least twenty percent of a longitudinal extension of the hollow main rod 12. In this embodiment, the flushing rod 20 is inserted along twenty percent of the longitudinal extension of the hollow main rod 12.

The opening 17 is substantially arranged at one end of the hollow main rod 12. At an opposite end of the hollow main rod 12 the tool end 14 of the surgical instrument 11 is arranged. The hollow main rod 12 comprises a widened grip portion 13 at the end of the hollow main rod 12 at which the opening 17 is arranged.

The hollow main rod 12 comprises a cavity 25 oriented in at least one operating state along a longitudinal extension 23 of the hollow main rod 12. The opening 17 is provided for insertion of the flushing rod 20 in a direction at least substantially parallel to a main extension direction of the cavity 25. During an actuation of the tool end 14, some parts of the main cavity are moved out of a direction along a main part of the longitudinal extension 23 of the hollow main rod 12. In this embodiment, the opening 17 is provided for inserting the flushing rod 20 in a direction parallel to the main extension direction of the cavity. A normal of the opening 17 is parallel to a normal of the cavity 25. The flushing rod 20 can be inserted into the hollow main rod 12 in an unbent state. It is conceivable, in a further embodiment of the system 10, to provide a bendable flushing rod 20 that is provided to be inserted in a direction which is non-parallel to the main extension direction of the cavity bent in order to be inserted into the hollow main rod 12.

A further opening 16 for connecting the surgical instrument 11 to a water conduit is arranged at the hollow main rod 12 of the surgical instrument 11. The further opening 16 is arranged at an end of the grip portion 13 which is opposite to the end of the grip portion 13 at which the opening 17 for inserting the flushing rod 20 is arranged. The further opening has a normal which is perpendicular to the normal of the cavity 25. During a flushing of the surgical instrument 11 in which the flushing rod 20 is used, the further opening 16 can be closed or can be left open to act as an outlet for flushing fluid and pollutants being flushed out.

The surgical instrument 11 comprises a knob 18 which is provided for operating the surgical instrument 11 and has an opening 19 for inserting the flushing rod 20 into the opening 17 of the hollow main rod 12. In order to operate the surgical instrument 11, the knob 18 is turned. The cable pulls 15 are attached to the knob 18 and are twisted by turning the knob 18. By this twisting, the tool end 14 changes its shape. The knob 18 is secured by a screw 24 to the grip portion 13 of the hollow main rod 12. The opening 19 of the knob 18 is arranged in a center of the knob 18 and is coaxial to the opening 17 of the hollow main rod 12.

The flushing rod 20 comprises a hollow rod 21 and a connection 22 which is provided for connecting the hollow rod 21 to a source of flushing fluid. The hollow rod 21 is made of stainless steel and has a diameter matching a width of the openings 17, 19. Water is used as a primary component of the flushing fluid. The flushing fluid can contain additional substances, such as decontaminants. The connection 22 has a funnel-like shape to provide a high pressure and a rapid flow of the flushing fluid.

A method for using the above-described system 10 comprises a step in which the flushing rod 20 is inserted into the hollow main rod 12 along at least twenty percent of the longitudinal extension 23 of the hollow main rod 12. The flushing rod 20 is inserted into the hollow main rod 12 via the opening 17 which is arranged at the end of the hollow main rod 12 and the opening 19 of the knob 18.

After inserting the flushing rod 20 into the hollow main rod 12, a flushing fluid is introduced into the hollow main rod via 12 the flushing rod 20 in a further method step. The flushing rod 20 is coupled to a source of the flushing fluid via the connection 22.

The method further comprises a step in which the flushing rod 20 is pulled out of the hollow main rod 12 while maintaining a stream of flushing fluid. Different parts of the hollow main rod 12 are subsequently flushed and cleaned while the flushing rod 20 is being pulled out. The flushing fluid and pollutants flushed out by the flushing fluid leave the hollow main rod 12 via the opening 17 after the flushing rod 20 is pulled out of the hollow main rod 12.

### REFERENCE NUMERALS

10 system
11 surgical instrument
12 hollow main rod
13 grip portion
14 tool end
15 cable pull
16 opening
17 opening
18 knob
19 opening
20 flushing rod
21 hollow rod
22 connection
23 longitudinal extension
24 screw
25 cavity

1. A system comprising a surgical instrument with a hollow main rod which has an opening, and comprising a flushing rod provided for flushing the hollow main rod and for being inserted at least partially into the hollow main rod through the opening.

2. The system according to claim 1, wherein the flushing rod is provided to be inserted along at least twenty percent of a longitudinal extension of the hollow main rod.

3. The system according to claim 1, wherein the opening is arranged substantially at one end of the hollow main rod.

4. The system according to claim 1, wherein the hollow main rod comprises a main cavity oriented in at least one operating state at least substantially along a longitudinal extension of the hollow main rod, and wherein the opening is provided for inserting the flushing rod in a direction which is at least substantially parallel to a main extension direction of the cavity.

5. The system according to claim 1, wherein the surgical instrument comprises a knob which is provided for operating the surgical instrument and has an opening for inserting the flushing rod into the opening of the hollow main rod.

6. The system according to claim 1, wherein the surgical instrument comprises cable pulls arranged in the hollow main rod.

7. The system according to claim 6, wherein the surgical instrument comprises a tool designed to change its shape upon being actuated by the cable pulls.

8. The system according to claim 1, wherein the flushing rod comprises a hollow rod and a connection provided for connecting the hollow rod to a source of a flushing fluid.

9. The system according to claim 8, wherein the connection has a funnel-like shape.

10. A flushing rod of a system according to claim 1.

11. The flushing rod according to claim 10, wherein the flushing rod comprises a hollow rod and a connection provided for connecting the hollow rod to a source of a flushing fluid.

12. A surgical instrument of a system according to claim 1.

13. The surgical instrument according to claim 12, wherein cable pulls are arranged in a hollow main rod.

14. The surgical instrument according to claim 13, further having a tool designed to change its shape upon being actuated by the cable pulls.

15. A knob for a surgical instrument of the system according to claim 5.

16. A method for using the system according to claim 1, comprising a step in which a flushing rod is inserted into a hollow main rod.

17. The method according to claim 16, wherein the flushing rod is inserted into the hollow main rod along at least twenty percent of a longitudinal extension of the hollow main rod.

18. The method according to claim 16, comprising a further step in which a flushing fluid is introduced into the hollow main rod via the flushing rod.

19. The method according to claim 18, comprising a further step in which the flushing rod is pulled out of the hollow main rod while maintaining a stream of flushing fluid.
